(19) United States
(12) Patent Application Publication
(10) Pub. No.: US 2014/0193386 A1
(43) Pub. Date: Jul. 10, 2014

(54) METHOD FOR ENZYMATIC CROSS-LINKING OF A PROTEIN

(71) Applicant: LifeBond Ltd., Caesarea (IL)

(72) Inventors: Orahn PREISS-BLOOM, Zichron Yakov (IL); Guy TOMER, Modi'in (IL); Nadav BRAMSON, Hadera (IL)

(73) Assignee: LIFEBOND LTD., Caesarea (IL)

(21) Appl. No.: 14/181,841

(22) Filed: Feb. 17, 2014

Related U.S. Application Data
(63) Continuation-in-part of application No. 12/999,632, filed on Dec. 20, 2010, now abandoned.
(60) Provisional application No. 61/129,321, filed on Jun. 18, 2008.

(51) Int. Cl. C12N 9/10

(52) U.S. Cl. CPC C12N 9/1044
USPC 424/94.5; 435/193; 435/68.1

(57) ABSTRACT

A method for cross-linking albumin for use as a sealant or glue for a biological system, for example to induce hemostasis and/or prevent leakage of any other fluid from a biological tube or tissue, such as lymph for example. The cross-linked albumin may optionally and preferably be applied as part of a bandage for example. In other embodiments, the present invention provides a method of enzymatically cross-linking globular proteins, by altering the structure of the protein to improve the accessibility of the protein to the cross-linking enzyme.

### FIELD OF THE INVENTION

The present invention relates to protein cross-linking, and more specifically to a method of improving enzymatic cross-linking of globular proteins using a structural modifier of the protein, and gels produced by this method.

### BACKGROUND OF THE INVENTION

Proteins which are able to undergo rapid cross-linking in situ are successfully utilized in a number of medical applications, such as in sealants and hemostats, in drug delivery, and in tissue engineering.

Nonetheless, existing products based on cross-linkable proteins have major flaws. For example, BioGlue® (CryoLife Inc., USA), a surgical adhesive composed of purified bovine albumin cross-linked by gluteraldhyde, is highly toxic and thus approved only for certain limited surgical applications, and not for general surgery.

Other synthetic and fibrin-based sealants are commonly used in the operating room, despite their low adhesive strength, which leads to significantly low efficacy.

The use of enzymes for cross-linking of certain proteins has previously been suggested, such as in PCT Application No. PCT/US07/25726, filed on Dec. 17, 2007 to some of the current inventors. A disadvantage of protein cross-linking by an enzymatic method is that such cross-linking reactions can only successfully be performed with proteins that contain reactive groups sufficiently accessible to the cross-linking enzymes. Examples of such proteins include casein, gelatin and wheat gluten.

Sealants comprising enzymatically cross-linked gelatin are known. Non-gelatin sealants have the advantage that, unlike gelatin, they can form solutions that remain in liquid phase at room temperature without the need for additives and thus will not necessitate any heating before use, such as in the operating room.

Furthermore, non-gelatin sealants can be much less viscous. The high viscosity of gelatin is also problematic for application via a surgical applicator nozzle. Spraying gelatin, even in solution, is very difficult, in contrast to spraying less viscous protein solutions, which is far easier.

Globular proteins, on the other hand, have a structure that makes their reactive groups insufficiently accessible for enzymatic protein cross-linking. Examples of globular proteins include β-lactoglobulin, α-lactalbumin, serum albumin and ovalbumin, recombinant human albumin and more, as described in PCT application PCT/NL05/000582, which describes the need for methods for improving accessibility of globular proteins for enzymatic cross-linking for industrial food manufacturing applications.

Several groups have improved the accessibility of globular proteins for enzymatic cross-linking for industrial food manufacturing applications. De Jong at al. (J. Agric. Food. Chem, 2001(49), 3389-3393) used microbial transglutaminase (mTG) to polymerize a 0.5% bovine serum albumin (BSA) solution that had been treated with dithiothreitol (DTT), a strong reducing agent. Nonaka et al (Agricultural and Biological Chemistry, 1989 (53, 10), 2619-2623) used mTG to polymerize a 1% BSA solution that has been treated with DTT. Both groups demonstrated mTG-catalyzed polymerization using SDS-PAGE but did not succeed in forming a mTG-crosslinked gel.

Kang et al (J Food Sci, 2003 (68, 7), 2215-2220) used BSA as an emulsifier in an oil in water emulsion, where the 5% w/v BSA emulsion was treated with mTG in the presence of 2-mercaptoethanol (2-ME) to form a BSA-stabilized emulsion gel. In that case, formation of the emulsion gels depended on the presence of 2-ME and enzymatic crosslinking was not essential for the gel formation process.

Gan et al (Food Hydrocolloids, 2009 (23), 1398-1405) used mTG alone or in combination with ribose followed by heat treatment (90° C. for 3 hours) to form BSA gels from solutions of 10% BSA. Gelation in that case was induced by the denaturative heat treatment rather than mTG crosslinking. This is a well known technique in the art as many globular food proteins, such as albumin, can be gelled (coagulated) by heat treatment (Tobitani et al, Macromolecules, 1997 (30, 17), 4845-4854.

Other denaturative methods of globular protein gelation have been described. Thiol-dependent gelation process involves denaturation by the reduction of intramolecular disulfide bonds and subsequent protein aggregation due to formation of new intermolecular hydrogen bonds or new intermolecular disulfide bonds as a result of thiol-disulfide exchange. BSA has 17 pairs of disulfide bonds and therefore is very responsive to thiol reducing agents. This has been described by Hirose at al. (J Food Sci, (55, 4), 915-917). Denaturative agents have been described for use in causing gelation of whey proteins including BSA. Xiong et al. used urea for this purpose (J. Agric Food Chem, 1990 (38, 10), 1887-1891).

Thus, denaturative treatment can independently induce non-enzymatic gelation of the above globular proteins, such that even in cases in which enzymes were used to apparently induce cross-linking, in fact cross-linking was induced through non-enzymatic mechanisms such as those described above.

Among treatments that are used to denature globular proteins are: incubation with reducing agents, incubation with denaturing or chaotropic agents, and heating. Denaturation by these methods can result in protein aggregation, completely independent of an enzymatic crosslinker, as a result of intermolecular bonds formed between protein regions. Such aggregation can be in the form of precipitation or gelation. With increasing concentrations of globular protein, more extensive denaturation is required. Increasing the intensity of the denaturative treatment increases the resultant gelation or precipitation of the protein independently of any enzymatic cross-linking.

### SUMMARY OF THE INVENTION

There is a need for, and it would be useful to have, a method for enzymatically cross-linking globular proteins, which is devoid of at least some of the limitations of the prior art.

The present invention overcomes these drawbacks of the background art by providing, in some embodiments of the present invention, a method for cross-linking globular proteins without the use of a denaturing pre-treatment that results in aggregation or gelation of the globular protein solution, such that cross-linking effectively occurs through the activity of the enzyme. By “effectively occurs” it is mean that crosslinking which leads to formation of a solid gel occurs due to activity of the enzyme, even if a slight amount of cross-linking occurs due to application of a denaturing treatment or pre-treatment.

Such a method is useful for a variety of medical applications including but not limited to the use as a sealant or glue for a biological system, for example to induce hemostasis and/or prevent leakage of any other fluid from a biological tube or tissue, such as lymph, bile, urinary tract or gastrointestinal tract for example. The cross-linkable albumin and cross-linker may optionally and preferably be applied together with a bioabsorbable or non-bioabsorbable backing or bandage. In another optional embodiment, the components are absorbed, adsorbed or otherwise adhered to or combined with the backing or bandage for example.

In other embodiments, the present invention provides a method of enzymatically cross-linking globular proteins, by altering the structure of the protein to improve the accessibility of the protein to the cross-linking enzyme.

According to some embodiments of the present invention, there is provided a method for cross-linking a globular protein, the method comprising adding to a solution of the globular protein a cross-linking enzyme and a structural modifier of the globular protein.

“Globular proteins” is used herein in its art-recognized meaning and includes proteins that have a globular domain. Preferably, however, aspects of the present invention relate to proteins that are strictly globular, which for example in this context may optionally relate to those proteins that cannot be cross-linked without the use of structure-modifying agents.

According to some embodiments of the present invention, there is provided a method for cross-linking a globular protein, the method comprising adding to a solution of the globular protein a cross-linking enzyme and a structural modifier of the globular protein.

According to some embodiments of the present invention, there is provided a method for preparing a medical gel, the method comprising adding to a solution of a cross-linkable globular protein a cross-linking enzyme and a structural modifier of the globular protein.

Optionally and preferably, the globular protein is modified in a manner that at least partially disrupts its tertiary structure (i.e. at least partially denatures it) while maintaining the protein in a soluble state such that the protein solution remains in liquid form.

According to some embodiments of the present invention, the globular protein comprises soy protein, conalbumin, bovine serum albumin (BSA), human serum albumin, recombinant human albumin, hemoglobin, ovalbumin, α-chymotrypsinogen A, α-chymotrypsin, trypsin, trypsinogen, β-lactoglobulin, myoglobin, α-lactalbumin, lysozyme, ribonuclease A, or cytochrome c, or combinations thereof.

Optionally and preferably, the globular protein comprises bovine serum albumin.

Optionally and preferably, the globular protein comprises human derived serum albumin.

Surprisingly the present inventors have found that cross-linking of globular proteins such as albumin with a crosslinking enzyme, such as mTG (microbial transglutaminase), resulting in gelation, is dependent upon three important factors: pH value of the solution containing the globular protein and the crosslinking enzyme (preferably between pH 6-9); a reducing agent; a chaotropic agent; and denaturation of the globular protein.

In previously submitted U.S. Ser. No. 12/999,632, filed on Dec. 20, 2010 and published as US 2011-0086014 on Apr. 14, 2011, which is owned in common with the present application and which has at least one inventor in common, TCEP (tris(2-carboxyethyl)phosphine hydrochloride) was previously reported to have an important effect on enabling crosslinking of albumin TCEP is a reducing agent so this effect was attributed to its activity as a reducing agent. However, TCEP also has an acidic pH and surprisingly, the present inventors have found that the previously reported effect of TCEP is due to its acidic pH as well as its reducing activity.

In particular, gelation occurred due to a combination of denaturation of BSA (albumin; the two terms are used interchangeably throughout the application), chaotropic agent, reducing agent, pH value between 6 and 9, and transglutaminase. In order to keep the BSA in solution, pH values between 6 and 9 are preferred, as otherwise precipitation may occur. The chaotropic agent is optionally urea. As described herein, urea is effective in a range of from 3M to 6M.

Optionally and preferably, denaturing of urea with heat and the chaotropic agent is performed before the reducing agent is combined with BSA in the presence of acidic pH. However, alternatively the reducing agent can be combined with BSA before chaotropic agent denaturing is performed.

According to at least some embodiments of the present invention, the effective pH range for mTG dependent gelation of denatured BSA at ambient temperatures in the presence of a reducing agent is between pH 3.5 and 4.5. Preferably the solutions are maintained in a temperature range of 22° C. to 37° C. for this pH range of values; in fact this pH range was shown to be effective for various conditions, including preferred urea concentrations and so forth.

It should be noted that wherever reference is made to “mTG” or “microbial transglutaminase”, any transglutaminase may optionally be considered. According to preferred embodiments of the present invention, the cross-linking enzyme comprises calcium independent microbial transglutaminase.

Preferably, the concentration of albumin for this type of solution is 1% to 10%.

As described herein any of the methods may optionally be used to produce a product, which may also optionally be used as a medical sealant or glue.

According to some embodiments there is provided use of cross-linked albumin as a medical sealant or glue. According to some embodiments there is provided use of albumin and a cross-linking enzyme as a medical sealant or glue.

According to some embodiments there is provided use of the medical sealant of in a medical application selected from the group consisting of reinforcement of surgical repair lines; provision of fluid-stasis; prevention of lymphorrhea; prevention of cerebro-spinal fluid (CSF) leakage; prevention of anastomotic dehiscence; and sealing of an attachment between a tissue and a material.

Optionally the medical sealant is provided in a form selected from the group consisting of a gel, a spray, a strip, a patch, and a bandage.

The composition as described herein may optionally be used for preparation of a tissue engineering scaffold. According to some embodiments there is provided use of enzyme cross-linked albumin as a tissue engineering scaffold.

According to some embodiments there is provided use of albumin and a cross-linking enzyme as a tissue engineering scaffold.

According to some embodiments there is provided use of the composition as described herein for preparation as a drug delivery platform.

According to some embodiments there is provided use of enzyme cross-linked albumin as a drug delivery platform.

According to some embodiments there is provided use of albumin and a cross-linking enzyme as a drug delivery platform.

According to some embodiments there is provided a method for cross-linking a globular protein, the method comprising adding to a solution of the globular protein a cross-linking enzyme and a structural modifier of the globular protein.

Prior to the herein described invention, enzyme-mediated polymerization of aqueous globular protein solutions has been demonstrated only at low protein concentrations, which require just mild denaturative treatment to denature globular proteins and facilitate enzyme crosslinking of the globular protein. These protein concentrations are too low to allow for the formation of a solid gel. At the higher protein concentrations that would be necessary to form a solid gel, higher levels of denaturative treatment have been used. The higher level of denaturative treatment itself, not enzymatic crosslinking, has resulted in aggregation or gelation of the protein.

As a result of the above-described technical limitations, the use of an enzymatically crosslinked globular protein gel has not previously been suggested for use in medical applications. The embodiments described herein overcome these limitations by enabling controlled crosslinking of globular proteins such as albumin with crosslinking enzymes such as transglutaminase.

According to some embodiments, there is provided the use of any of the methods described herein in the preparation as a medical sealant.

According to some embodiments of the present invention, there is provided the use of cross-linked albumin as a medical sealant or glue.

According to some embodiments of the present invention, there is provided the use of albumin and a cross-linking enzyme as a medical sealant or glue.

According to some embodiments, there is provided the use of the medical sealant of the present invention in a medical application selected from the group consisting of reinforcement of surgical repair lines; provision of fluid-stasis; prevention of lymphorrhea; prevention of cerebro-spinal fluid (CSF) leakage; prevention of anastomotic dehiscence; and sealing of an attachment between a tissue and a material.

Optionally and preferably, the medical sealant is provided in a form selected from the group consisting of a gel, a spray, a strip, a patch, and a bandage.

According to some embodiments, there is provided the use of the methods of the present invention for preparation of a tissue engineering scaffold.

According to some embodiments of the present invention, there is provided the use of cross-linked albumin as a tissue engineering scaffold.

According to some embodiments of the present invention, there is provided the use of albumin and a cross-linking enzyme as a tissue engineering scaffold.

According to some embodiments, there is provided the use of the method of the present invention for preparation as a drug or polypeptide (or other biological) delivery platform. It should be noted that when reference is made to a “drug” any therapeutic agent is considered to be encompassed thereby, including but not limited to small molecules, large macromolecules such as macrolides, taxanes such as paclitaxel, polypeptides of any type whether linear or cyclic (such as cyclosporine, for example), peptides of any type, nucleotide-based agents and so forth.

According to some embodiments of the present invention, there is provided the use of cross-linked albumin as a drug or polypeptide delivery platform.

According to some embodiments of the present invention, there is provided the use of albumin and a cross-linking enzyme as a drug or polypeptide delivery platform.

According to a preferred embodiment, the globular protein solution or enzyme is preferably prepared for medical use.

According to some embodiments, the globular protein solution or enzyme are treated to reduce impurities, including but not limited to purities related to micro-organisms. According to a preferred embodiment, the microbial colony forming unit (CFU) count of the globular protein solution or enzyme composition is reduced or eliminated through such treatment.

In a preferred embodiment, such treatment optionally comprises sterilizing the globular protein solution or enzyme composition through sterile filtration and/or radiation sterilization (gamma or ebeam).

According to a preferred embodiment, the endotoxin level of the globular protein solution or enzyme composition is reduced or eliminated. Optionally and preferably, the endotoxin level of the enzyme composition is reduced through cation exchange chromatography.

Unless defined otherwise, all technical and scientific terms used herein have the same meaning as is commonly understood by one of skill in the art to which this invention belongs. All patents, patent applications, and publications mentioned herein are incorporated herein by reference.

“Wound” as used herein refers to any damage to any tissue of a patient that results in the loss of blood from the circulatory system or the loss of any other bodily fluid from its physiological pathway. The tissue can be an internal tissue, such as an organ or blood vessel, or an external tissue, such as the skin. The loss of blood or bodily fluid can be internal, such as from a ruptured organ, or external, such as from a laceration. A wound can be in a soft tissue, such as an organ, or in hard tissue, such as bone. The damage may have been caused by any agent or source, including traumatic injury, infection or surgical intervention. The damage can be life-threatening or non-life-threatening.

“TG” refers to transglutaminase of any type; “mTG” may also refer to microbial transglutaminase and/or to any type of transglutaminase, depending upon the context (in the specific experimental Examples below, the term refers to microbial transglutaminase).

“Gel” refers to a substantially dilute crosslinked system, which exhibits no flow when in the steady-state. It may also refer to the phase of a liquid achieved when a three-dimensional crosslinked network within the liquid develops, causing the elastic modulus of the composition to become greater than its viscous modulus.

“Denaturing agents” refers to substances that affect the structure of proteins. This group includes but is not limited to reducing agents, which disrupt the disulfide bonds in proteins, and chaotropic agents, which disrupt the hydrogen bonds in proteins.

As used herein, “about” means plus or minus approximately ten percent of the indicated value.

Other features and advantages of the invention will be apparent from the following detailed description, and from the claims.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in order to provide what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 shows an exemplary embodiment of an exemplary method according to the present invention for cross-linking albumin; and

FIG. 2 shows an exemplary embodiment of another exemplary method according to the present invention for cross-linking albumin.

### DESCRIPTION OF THE PREFERRED EMBODIMENTS

According to some embodiments of the present invention, there is provided a composition comprising a disulfide bond disrupting agent to prevent aggregation or gelation of a denatured globular protein in order to keep the denatured protein in solution. Denaturation may optionally be caused for example by a hydrogen bond disrupting method (heat and/or denaturing agent) at concentrations that would normally cause aggregation (precipitation or gelation) of a globular protein solution. This enables the formation of a solid protein gel through enzymatic crosslinking. As used herein the term “enzymatic crosslinking” refers to cross-linking which is functionally due to the effect of the enzyme, as opposed to the effect of denaturation itself. The resultant gel has many applications, including but not limited to applications in the medical and food industries as described herein.

Some non-limiting examples of these applications are given below. For example, the composition may optionally be used for the sealing of a vascular graft such as Dacron vascular graft in acute aortic dissection; as an adjunct to controlling air leaks on the lung parenchyma following resection; as a matrix for immobilization of cells or enzymes for in vivo, in vitro or ex vivo use as a bioreactor or biosensor; as a biomimetic scaffold for tissue engineering; as a platform for drug delivery; for preparation of albumin microspheres for controlled release of drugs; to construct a contact lens or other ophthalmic device; for artificial skin; as a wound dressing; as a surgical sealant along suture lines or about surgical staples, forming an anastomosis (the sutures or staples can be used, e.g., to join blood vessels, bowels, ureter, or bladder); for controlling or arresting organ bleeding; for coating the lumenal surface of a blood vessel, or other tissue cavity that has been damaged by trauma, surgical intervention, angioplasty or disease; for soft tissue augmentation or replacement applications including facial tissue augmentation, urinary incontinence; for plastic surgery reconstruction; as a spinal disc replacement; for lung volume reduction; to form albumin sponges and albumin-coated bandages for bleeding arrest and wound healing; to form electrospun albumin fibers; and to form albumin beads as packing material for HPLC columns and other chromatography applications.

Some non-limiting food industry applications include: encapsulation of food ingredients in albumin microspheres or nanospheres for: protection against oxidation, retention of volatile ingredients, taste masking, enhanced stability; gelation of food products, such as yoghurt-desserts; and stabilization of emulsions by acting as emulsifier to create emulsion gels.

According to other embodiments of the present invention, there is provided an enzyme crosslinked solid globular protein gel for use in medical applications. All prior art is not appropriate for medical applications as a gel either because no gel was formed or because one or more non biocompatible materials were used.

According to still other embodiments of the present invention, there is provided an in situ enzymatic crosslinked globular protein gel, optionally and preferably cross-linked when in contact with the tissue of a subject, more preferably comprising a denaturing agent as described above. Optionally, this gel is not treated with heating.

According to some embodiments of the present invention, there is provided a method for cross-linking albumin for use as a sealant or glue for a biological system, for example to induce hemostasis and/or prevent leakage of any other fluid from a biological tube or tissue, such as lymph for example. The cross-linked albumin may optionally and preferably be applied as part of a bandage for example.

In other embodiments, the present invention provides a method of enzymatically cross-linking globular proteins, by altering the structure of the protein to improve the accessibility of the protein to the cross-linking enzyme.

According to some embodiments of the present invention, there is provided a method for cross-linking a globular protein, the method comprising adding to a solution of the globular protein a cross-linking enzyme and a structural modifier of the globular protein.

According to further embodiments of the present invention, there is provided a method for preparing a medical gel, the method comprising adding to a solution of a cross-linkable globular protein a cross-linking enzyme and a structural modifier of the globular protein.

According to some embodiments of the present invention, the structural modifier is a reducing agent.

The reducing agent is used to disrupt the disulfide bonds that are responsible for maintenance of the globular structure of globular proteins. By disrupting these disulfide bonds, and thereby modifying the structure of the globular protein, the cross-linker substrate sites on the protein are exposed.

Non-limiting examples of suitable reducing agents that can be used to disrupt the disulfide bonds of globular proteins include hexafluoroisopropanol (HFIP), dimethyl sulfoxide (DMSO), sodium dodecyl sulfate (SDS), glutathione, hydroquinone, 2-mercaptoethanol, 2-mercaptoethylamine, tris(2-carboxyethyl)phosphine hydrochloride (TCEP), cysteine, dithiothreitol (DTT), and N-ethylmaleimide.

Globular proteins which can be cross-linked in accordance with the principles of the present invention include, for example, soy protein, conalbumin, bovine serum albumin (BSA), human serum albumin, recombinant human albumin, hemoglobin, ovalbumin, α-chymotrypsinogen A, α-chymotrypsin, trypsin, trypsinogen, β-lactoglobulin, myoglobin, α-lactalbumin, lysozyme, ribonuclease A, and cytochrome c.

The cross-linking enzyme of the present invention is optionally and preferably transglutaminase (TG), which may optionally comprise any type of calcium dependent or independent transglutaminase (mTG), such as, for example, a microbial transglutaminase.

Optionally, newly available commercial transglutaminase products containing 10% or more mTG may be used. Non-limiting examples of commercially available transglutaminase products of this sort include those produced by Ajinomoto Co. (Kawasaki, Japan) and Yiming Chemicals (China). A preferred example of such a product from this company is the Activa TG—Ingredients: mTG and maltodextrin; Activity: 810-1,350 U/g of Activa. Preferred products from Yiming include one product containing 10% mTG and 90% maltodextran and one product containing 10% mTG and 90% lactose, also of activity 810-1,350 U/g of product.

According to a preferred embodiment of the present invention, microbial transglutaminase (mTG) is used as the cross-linker and albumin as the protein. The mTG-substrates in the albumin that are exposed in accordance with this method are glutamine and lysine residue.

It should be noted that the specificity of microbial transglutaminase differs from that of tissue transglutaminase with regards to its ability to cross-link albumin Some tissue transglutaminases are natively able to cross-link albumin to some extent. However, cross-linking of albumin using mTG requires treating the albumin with a reducing agent (de Jong G A, et al. J. Agric. Food Chem., 49 (7), 3389-3393, 2001.).

Cross-linked globular proteins prepared in accordance with the method of the present invention have a wide range of applications for medical use, such as for a novel surgical sealant, tissue adhesive and hemostat. Other uses include tissue scaffolds and cell scaffolds.

Once the globular structure of a globular protein, such as albumin, has been disrupted and its mTG-substrates exposed, it provides an ideal protein for the preparation of adhesive compositions for use in soft tissue applications.

According to some embodiments there is provided a composition comprising a cross-linkable globular protein, a cross-linking enzyme and a structural modifier of the globular protein, for use in medical applications.

According to some embodiments, a gel prepared according to any of the methods of the present invention may be used in a medical application, such as for example, a medical sealant.

A medical sealant prepared according to the method of the present invention may be used, for example, in reinforcement of surgical repair lines (including staple lines or suture lines); for providing fluid-stasis, such as hemostasis or lymphostasis; for preventing lymphorrhea; for prevention of cerebro-spinal fluid (CSF) leakage; for preventing anastomotic dehiscence; or for sealing an attachment between a tissue and a material, including another tissue, an implant, a prosthesis, or a skin graft.

The medical sealant may be provided, for example, in the form of a gel, a foam, a spray, a strip, a patch, or a bandage.

According to some embodiments of the present invention, the composition is provided in a bandage, which is preferably adapted for use as a hemostatic bandage. The herein described compositions may additionally have one or more uses including but not limited to tissue adhesives (particularly biomimetic tissue adhesives), tissue culture scaffolds, tissue sealants, hemostatic compositions, drug delivery platforms, surgical aids, or the like, as well as other non-medical uses, including but not limited to edible products, cosmetics and the like, such as, for example, in purification of enzymes for use in food products.

### EXAMPLES

Reference is now made to the following examples, which together with the above description, illustrate some embodiments of the invention in a non limiting fashion. Unless otherwise indicated, all of the below Examples relate to one or more of the below list of Materials.

### Materials:

Bovine Albumin Fraction V 96%-99% [Biological Industries, Israel, Lot#700118], Sodium Acetate (x3H2O C.P. lot #104013), Dulbecco's Phosphate Buffered Saline without Calcium and Magnesium [Biological Industries, Israel], microbial Transglutaminase ACTIVA-TG 10% enzyme powder in maltodexterin [Ajinomoto, Japan], urea (Sigma), TCEP (Sigma), 2-mercaptoethanol (Aldrich chemicals), collagen strips (Nitta Casings) and NaCl (Frutarom, Israel).

### Example 1

### Effect of TCEP Concentration on Physical State of BSA

Example 1 shows that inclusion of TCEP at a concentration greater than at least 12 mM can prevent heat- and urea-induced aggregation or gelation of a 5% BSA solution, thereby demonstrating the overall efficacy of TCEP as a gelation controlling agent. However, as described below, this effect may be at least partially attributed to pH values of the resultant solution, as TCEP is acidic.

5% w/w BSA containing 5.6M urea was mixed with various concentrations of TCEP followed by heating at 70° C. for 10 minutes. The physical state of the BSA+urea+TCEP solution after the heating step is described in the table below.

TABLE 1
TCEP effect

| TCEP | Physical state of the BSA + urea + TCEP |
|---|---|
| concentration (mM) | solution after heating at 70° C. for 10 minutes |
| 50 | Clear liquid |
| 25 | Clear liquid |
| 20 | Clear liquid |
| 15 | Clear liquid |
| 12.5 | Clear liquid |
| 10 | Viscous clear liquid |
| 7.5 | Very Viscous clear liquid - almost a gel |
| 5 | Transparent gel |
| 2.5 | Opaque gel |
| 0 | White solid |

### Example 2

### Effect of Heating and Various Combination of BSA, Urea and TCEP Concentration on the Physical State of BSA

Example 2 shows that the physical state of an albumin solution that has been heat treated for 10 minutes at 70° C. shows a direct concentration dependence on urea and TCEP (greater amounts of urea and TCEP result in a more liquid state) and on the concentration of albumin in an inverse correlation (more albumin results in aggregation/gelation). Without wishing to be limited by a single hypothesis, it may be the molar ratio of urea and TCEP to albumin that determines the physical state.

BSA solutions containing urea were heated for 10 minutes at 70° C. in the presence of TCEP and the physical state of the solution was recorded.

TABLE 2
TCEP/Urea vs Protein Concentration

| | | | Physical state |
|---|---|---|---|
| | | | after 10 min at |
| BSA % | Urea conc. (M) | TCEP conc. (mM) | 70° C. |
| 16 | 4.5 | 50 | Clear gel |
| 16 | 4.5 | 10 | Clear gel |
| 16 | 4.5 | 2 | White solid |
| 16 | 2.25 | 50 | Clear gel |
| 16 | 2.25 | 10 | White solid |
| 16 | 2.25 | 2 | White solid |
| 16 | 1.125 | 50 | Clear gel |
| 16 | 1.125 | 10 | White solid |
| 16 | 1.125 | 2 | White solid |
| 8 | 4.5 | 50 | Clear liquid |
| 8 | 4.5 | 10 | Clear gel |
| 8 | 4.5 | 2 | White solid |
| 8 | 2.25 | 50 | Clear gel |
| 8 | 2.25 | 10 | Clear gel |
| 8 | 2.25 | 2 | White solid |
| 8 | 1.125 | 50 | Clear gel |
| 8 | 1.125 | 10 | White solid |
| 8 | 1.125 | 2 | White solid |
| 4 | 4.5 | 50 | Clear liquid |
| 4 | 4.5 | 10 | Clear liquid |
| 4 | 4.5 | 2 | Clear gel |
| 4 | 2.25 | 50 | Clear liquid |
| 4 | 2.25 | 10 | Clear semi-gel |
| 4 | 2.25 | 2 | White solid |
| 4 | 1.125 | 50 | Clear liquid |
| 4 | 1.125 | 10 | Clear semi-gel |

### Example 3

### Effect of TCEP Concentration on mTG Dependent Gelation of BSA

This Example shows that disulfide bond reducing agents have an inhibitory effect on microbial transglutaminase, although the effect of TCEP was at least partially due to pH values as it is acidic.

500 ul 4% w/w solution containing 4.5M urea was heated at 70° C. for 3 minutes until precipitation occurred. Next, 25 ul of TCEP at various concentrations was added and the effect on the precipitated material was recorded. Next, 100 ul of 0.75% w/w mTG (7.5% w/w ACTIVA-TG 10%) was added to reactions that were clarified by the addition of TCEP and the reactions were incubated at 37° C.

TABLE 3
TCEP inhibition of gelation

| TCEP concentration | Physical state after | After incubation |
|---|---|---|
| (mM) | adding TCEP | with 100 ul mTG |
| 50 | Clarified immediately | Did not gel |
| 25 | Clarified immediately | Did not gel |
| 10 | Clarified after 5 | Soft gel after 75 min |
| | minutes | |
| 5 | Hardly clarified | Not determined |
| 0 | Did not clarify | Not determined |

### Examples 4 and 5

### Effect of Manipulating Thiol Bond Formation

Examples 4 and 5 relate to the effect of manipulating thiol bond formation in terms of increasing or decreasing gelation. DTT and 2-mercaptoethanol trigger heat-induced protein aggregation in the presence of urea at temperatures where urea alone or reducing agent alone do not cause aggregation, e.g. 50° C.

The aggregation, precipitation and gelation that occur as a result of incubation with disulfide bond reducing agents such as DTT and 2-mercaptoethanol may be prevented through the addition of salt (Lee et al, Agricultural and Biological Chemistry, Vol. 55, No. 8 (1991) pp. 2057-2062). The salt may prevent intermolecular interaction of disulfide-reduced protein. The results below relate to mTG-dependent gelation of a solution of BSA that has been denatured with a combination of heating, urea and 2-ME (2-mercaptoethanol), and optionally salt.

### Example 4

### Prevention of Thiol-Induced Gelation of BSA by Salt

A solution of 10% w/w BSA and 5.6M urea was incubated with 2-mercaptoethanol and NaCl in various combinations at different temperatures. The physical state of the solution after heating was recorded. Tables 4 and 5 show that there is a clear concentration dependence of gelation on the concentration of salt; however, treatment with sufficient heating could overcome the presence of salt.

TABLE 4
Effect of salt on gelation

| | | | 50° C., 10 | 60° C., 10 |
|---|---|---|---|---|
| Reaction # | 2-mercaptoethanol | NaCl | minutes | minutes |
| 1 | — | — | Clear liquid | White solid |
| 2 | — | 400 mM | Clear liquid | Clear solid |
| | | | | gel |
| 3 | 50 mM | — | White solid | White solid |
| 4 | 50 mM | 400 mM | Clear | White solid |
| | | | viscous | |
| | | | liquid | |

TABLE 5
effect of different salt concentrations on gelation

| Reaction # | 2-mercaptoethanol | NaCl | 50° C., 25 minutes |
|---|---|---|---|
| 1 | 2 mM | — | Opaque gel |
| 2 | 2 mM | 62.5 mM | Clear gel |
| 3 | 2 mM | 125 mM | Clear gel |
| 4 | 2 mM | 250 mM | Clear liquid |
| 5 | 2 mM | 500 mM | Clear liquid |

### Example 5

### mTG- and urea-Dependent Gelation Using 2-Mercaptoethanol as Reducing Agent

Reactions with Urea
Solution 1: 8.33% BSA w/w, 4.67M urea, 500 mM NaCl, 1.67 mM 2-mercaptoethanol
Solution 2: 8.33% BSA w/w, 4.67M urea, 250 mM NaCl, 1.67 mM 2-mercaptoethanol
The solutions were heated at 50° C. for 25 minutes. They were both clear liquid.
Reaction A: To 500 ul of solution 1, 125 ul water was added.
Reaction B: To 500 ul of solution 1, 125 ul 1.4% w/w mTG (14% w/w ACTIVA-TG 10%) was added.
Reaction C: To 500 ul of solution 2, 125 ul water was added.
Reaction D: To 500 ul of solution 2, 125 ul 1.4% w/w mTG (14% w/w ACTIVA-TG 10%) was added.
Reactions A through D were incubated at 40° C. for 3 hours.
Reactions B and D were both turned to gels. The control reactions A and C were liquid.
Solution 3: 8.33% BSA w/w, 4.67M urea, 8.33 mM 2-mercaptoethanol, 500 mM NaCl
Solution 4: 8.33% BSA w/w, 4.67M urea, 8.33 mM 2-mercaptoethanol
The solutions were heated at 50° C. for 15 minutes. Solution 4 turned to opaque gel. Solution 3 remained a clear liquid.
Reaction E: To 500 ul of solution 3, 125 ul water was added.
Reaction F: To 500 ul of solution 3, 125 ul 1.4% w/w mTG (14% w/w ACTIVA-TG 10%) was added
Reactions E and F were incubated at 40° C. After 3 hours both reactions were a viscous liquid. The reactions were then incubated at room temperature for 62 hours and both turned to gel.
Reaction without Urea:
Solution 5: 8.33% BSA w/w, 1.67 mM 2-mercaptoethanol, 500 mM NaCl
Solution 6: 8.33% BSA w/w, 1.67 mM 2-mercaptoethanol, 250 mM NaCl
Solution 7: 8.33% BSA w/w, 1.67 mM 2-mercaptoethanol

### Solution 8: 8.33% BSA w/w

The solutions were heated at 50° C. for 32 minutes and then at 60° C. for 38 minutes. They were all clear liquid.

To 500 ul of solutions 5 to 8, 125 ul 1.4% w/w mTG (14% w/w ACTIVA-TG 10%) was added. The reactions were incubated at 40° C. for but did not gel after 20 hours incubation.

Solutions 5-8 were also heated at 70° C. for 30 minutes. Reactions 7 and 8 precipitated, but reactions 5 and 6 remained each a clear liquid.

To 500 ul of solutions 5 and 6 that were heated at 70° C., 125 ul 1.4% w/w mTG (14% w/w ACTIVA-TG 10%) was added. The reactions were incubated at 40° C. for but did not gel after 20 hours incubation.

These results demonstrate that mTG-induced gelation of BSA in the presence of 2-mercaptoethanol can be achieved by inclusion of urea. Without urea higher concentrations of 2-mercaptoethanol and higher temperatures are required in order to denature the protein, but the mTG enzyme might be sensitive to higher concentrations of 2-mercaptoethanol. Also, higher concentration of 2-mercaptoethanol may result in mTG-independent gelation as demonstrated by reactions E and F. The results also demonstrate that NaCl inhibits physical precipitation/gelation of denatured BSA induced by treatment with heat, urea and mercaptoethanol thus allows addition of mTG for enzyme dependent gelation. It should be noted that in this experiment the mTG used was not purified and therefore EDG may not be as efficient as adding purified mTG in the Examples below.

### Example 6

### Cross-Linking of Albumin

This Example shows that unmodified globular proteins, such as bovine albumin do not undergo cross-linking in the presence of microbial transglutaminase (mTG), either in sodium acetate or Dulbecco's phosphate buffered saline buffers, thereby demonstrating that this inability to undergo cross-linking is not affected by the choice of buffer.

0.1M Sodium Acetate solution at pH 6.0 was prepared. 0.075% mTG solution in sodium acetate buffer was prepared.

25% (w/w) albumin solutions were prepared into the following solutions:

Solution A—bovine albumin in 0.1M sodium acetate solution.

Solution B—bovine albumin in 0.1M Phosphate Buffered Saline solution.

### Results:

Table 6 below shows cross-linking times of albumin using microbial transglutaminase. Cross-linking is defined as the time in which a massive gelatinous mass is formed.

As illustrated in the table, 25% w/w albumin in 0.1M Phosphate Buffered Saline buffer or in 0.1M sodium acetate buffer does not form cross-links in the presence of mTG. No cross-linking occurred in 2 hours after the albumin solution. Solutions were examined the following morning and still no cross-linking had occurred.

TABLE 6

| | | Cross-linking Time | |
|---|---|---|---|
| Solution | Test # | (min) | Description of Cross-Linked Gel |
| A | 1 | Did not cross-link | Solution did not cross-link and |
| | | | remained liquid for the duration |
| | | | of the test (2 hours) |
| A | 2 | Did not cross-link | Solution did not cross-link and |
| | | | remained liquid for the duration |
| | | | of the test (2 hours) |
| A | 3 | Did not cross-link | Solution did not cross-link and |
| | | | remained liquid for the duration |
| | | | of the test (2 hours) |
| B | 1 | Did not cross-link | Solution did not cross-link and |
| | | | remained liquid for the duration |
| | | | of the test (2 hours) |
| B | 3 | Did not cross-link | Solution did not cross-link and |
| | | | remained liquid for the duration |
| | | | of the test (2 hours) |
| B | 3 | Did not cross-link | Solution did not cross-link and |
| | | | remained liquid for the duration |
| | | | of the test (2 hours) |

### Example 7

### The Effect of TCEP is Due to its Acidic pH as Well as its Reducing Activity

### Materials

Reagents: BSA (Biological Industries, Israel), 0.5M neutral TCEP pH 7 (Sigma catalogue number 646547, LOT#: MKBP0515V), acidic TCEP (Sigma catalogue number C4706)

### Methods

Four tubes were filled each with 1 ml of 5% BSA in 4.5M urea. The tubes were heated for 10 minutes at 70° C., a large white aggregate was formed, which was dispersed by vortexing to become a turbid suspension. Different solutions were added to each tube as follows:

Tube #1: 110 μl neutral 0.5M TCEP (pH7)+30 μl water.

After addition, the tube content remained a turbid suspension. Addition of 100 μl 1N HCl and 20 μl concentrated HCl (32% w/w) turned the turbid suspension to a clear solution, pH was <2

Tube #2: 30 μl 1N HCl+100 μl water. The tube content became a turbid gel. Addition of 20 μl concentrated HCl (32% w/w) did not dissolve the gel.

Tube #3: 110 μl neutral 0.5M TCEP (pH7)+30 μl 1N HCl. Tube content remained a turbid suspension. Addition of 20 μl concentrated HCl (32% w/w) turned the turbid liquid suspension to a clear solution, pH was 2-2.5

Tube #4: 110 μl acidic 0.5M TCEP+30 μl water. The turbid suspension dissolved and became a clear solution. The results are shown in Table 7 below.

TABLE 7
Effect of pH and Reducing Agent

| | 1^{st} addition | | | 2^{nd} addition | |
|---|---|---|---|---|---|
| | Reducing | | Physical | | Physical |
| Tube # | agent | acid | description | acid | description |
| 1 | 48 mM | | Turbid | 241 mM | Clear |
| | TCEP, | | suspension | HCl | solution, pH |
| | pH 7 | | | | <2 |
| 2 | | 26 mM | Turbid gel | 176 mM | Turbid gel |
| | | HCl | | HCl | (no change) |
| 3 | 48 mM | 26 mM | Turbid | 176 mM | Clear |
| | TCEP, | HCl | suspension | HCl | solution (pH |
| | pH 7 | | | | 2-2.5) |
| 4 | 48 mM | | Clear solution, | — | — |
| | TCEP, | | pH 3-3.5 | | |
| | acidic | | | | |

The experiment described above shows that in order to keep urea and heat denatured BSA in solution both reducing agent (TCEP) and acidic pH are required.

### Example 8

### Effect of pH on Denatured BSA Physical State and EDG (Enzyme Dependent Gelation)

Materials (are as above).

### Methods:

30 ml of 5% BSA in 4.5M urea were heated at for 10 minutes at 70° C. until a large white aggregate was formed. Then 3 ml of 0.5M acidic TCEP were added and the aggregate dissolved resulting in a clear solution. 600 μl were removed and replaced with 600 μl 0.5N NaOH, at each point the pH was monitored with a pH meter. The 600 μl were split to 2 tubes with 300 μl each. 15 μl of mTG (about 700 IU/ml, in 20 mM sodium citrate, pH 6.0) were added to one tube, 15 μl of mTG buffer (20 mM sodium citrate, pH 6.0) were added to the other tube, and the tubes were incubated at 37° C., and monitored for gelation by inverting the tubes from time to time. The results are shown in Table 8 below.

TABLE 8
Effect of pH on Gelation

| Amount of | | | |
|---|---|---|---|
| 0.5N NaOH | | | |
| added | | | |
| (accumulated) | pH | +mTG | +buffer |
| — | 3.24 | — | — |
| 600 μl | 3.52 | Liquid at 20 | Liquid at 20 min & 60 min |
| | | min & 60 min | |
| 1200 μl | 3.68 | Liquid at 20 | Liquid at 20 min & 60 min |
| | | min & 60 min | |
| 1800 μl | 3.83 | Gel at 20 min | Liquid at 20 min & 60 min |
| 2400 μl | 4.0 | Gel at 20 min | Viscous liquid at 20 min, gel at |
| | | | 60 min |
| 3000 μl | 4.13 | — | — |
| 3600 μl | 4.31 | — | — |
| 4200 μl | 4.39 | — | — |
| | (gel) | | |

The above experiment demonstrates that pH is a key parameter that determines whether a denatured BSA solution can be gelled by mTG through crosslinking. Furthermore, the above experiment demonstrates that the pH is a key parameter for two different mechanisms. First, the solution pH determines its physical state. At pH<4.3 the BSA solution is in a liquid state, whereas at pH>4.3 the BSA in solution undergoes physical gelation. At a pH range just below the physical gelation point, the solution might physically gel at elevated temperatures as is shown at pH 4.0 for the BSA+buffer, even without mTG.

Without wishing to be limited by a single hypothesis, the effect of pH on gelation (with or without mTG) may be related to the isoelectric point (pI) of BSA which occurs at pH 4.7. At the pI, the electrostatic charge on the protein molecules is reduced to the greatest extent, thus maximizing the attraction forces between the molecules which may increase the protein tendency to precipitate or gel. At the more acidic pH range, the pH value is further from the protein pI, and the electrostatic charges repel each other, preventing precipitation or gelation. In addition, mTG shows the highest enzymatic activity between pH 5 and 9 (Ando H et al, 1989, Agric. Biol. Chem 53: 2613-7). Below pH 3.5 the enzymatic activity of mTG is inhibited. Therefore, the effective pH range for mTG dependent gelation of urea and heat denatured BSA at ambient temperatures is between pH 3.5 and 4.5. In fact this pH range holds for at least the temperature range of 22° C. to 37° C. (some data in other Examples, other data not shown).

### Example 9

### Effect of DTT and HCl Concentration

The effect of DTT (dithiothreitol) on solubilization of heat and urea denatured BSA aggregate was studied and it was found that DTT has a greater effect when it is added to the BSA/urea solution before the heat treatment rather than after heat treatment.

Materials are all as previously described. BSA solution was prepared as previously described. 5% BSA solution was prepared in 4.5M urea, pH 5.0. 0.5 ml of this solution was placed in an Eppendorf tube to which the reducing agent was added. Next the solution was subjected to 70° C. temperature for 8 min. Next HCl or NaOH was added to the material. The results are shown below in Table 9.

TABLE 9
Effect of DTT and HCl Concentration

| | | | |
|---|---|---|---|
| 1 | No reducing agent | 10 μl HCl | Transparent gel |
| | | (32%) | |
| 2 | 50 mM DTT | 10 μl HCl | Clear solution |
| | | (32%) | |
| 3 | 50 mM DTT | 50 μl HCl (32%) | Clear + undissolved |
| | | | lump, clarified after a |
| | | | while |
| 4 | 50 mM DTT | 1 μl HCl (32%) | Viscous white liquid |
| 5 | 25 mM DTT | 10 μl HCl | Clear solution |
| | | (32%) | |
| 6 | 10 mM DTT | 10 μl HCl | White gel |
| | | (32%) | |
| 7 | 5 mm DTT | 10 μl HCl | White gel |
| | | (32%) | |
| 8 | 50 mM DTT | 10 μl NaOH | Clear solution (pH 9-10) |
| | | (4N) | |
| 9 | No reducing agent | 10 μl NaOH | Transparent gel |
| | | (4N) | |

The above Table 9 shows that solubilization of heat and urea denatured BSA can be affected by both DTT and HCl concentration (the latter being a determinant of pH). The minimal DTT concentration which has this effect is 25 mM or 0.5 mmol/gram BSA.

Table 9 shows also that solubilization of heat/urea denatured BSA can be achieved also by addition of NaOH which increases the solution pH. DTT also caused such solubilization to occur after the addition of NaOH. Without wishing to be limited by a single hypothesis, solubilization of heat/urea denatured BSA at an increased pH is probably due increased repelling forces between protein molecules at a pH which is further from the isoelectric point.

Interestingly a similar effect is seen at two different pH ranges. If DTT is present in a concentration above 10 mM with a denaturing temperature in a range of from 50° C. to 90° C., then the two effective pH ranges are pH>9 or pH<4.

### Example 10

### Enzyme Dependent Gelation of Solubilized Heat/Urea Denatured BSA at Alkaline pH

Materials are as given previously.

Method

30 ml of 5% BSA in 4.5M urea were heated in the presence of a reducing agent (either 25 mM DTT, 25 mM TCEP pH 7, or 50 mM mercaptoethanol) for 10 minutes at 70° C. until a large white precipitate formed. After cooling to room temperature, 600 μl of 4N NaOH were added and solution was stirred until no white aggregates were left. 20 μl of concentrated HCl (32%) were added while constantly monitoring pH.

At certain pH values 2×300 μl samples were moved to separate tubes, and 5 μl mTG or mTG buffer were added to each tube, followed by incubation at 37 deg. Gelation was determined by cessation of flow inside the tubes. A similar method was performed with TCEP and also with beta-mercaptoethanol. Table 10 shows the effect of pH on physical state and mTG dependent gelation of BSA denatured with heat, urea and DTT. Table 11 shows the effect of pH on physical state and mTG dependent gelation of BSA denatured with heat, urea and TCEP.

TABLE 10
enzyme dependent gelation with DTT

| Amount of | | | |
|---|---|---|---|
| 32% HCl | | | |
| added | | | |
| (accumulated) | pH | +mTG | +buffer |
| — | 10.74* | Liquid after 3 hr | Liquid after 3 hr |
| 20 μl | 10.52 | — | — |
| 40 μl | 10.36 | — | — |
| 60 μl | 10.15* | Liquid after 3 hr | Liquid after 3 hr |
| 80 μl | 9.87* | Liquid after 3 hr | Liquid after 3 hr |
| 100 μl | 9.67 | — | — |
| 120 μl | 9.35* | Liquid after 3 hr | Liquid after 3 hr |
| 140 μl | 9.04 | — | — |
| 160 μl | 8.72* | Gel at 10 min | Liquid after 3 hr |
| 180 μl | 8.37 | | |
| 200 μl | 8.02* | Gel at 10 min | Liquid after 3 hr |
| 220 μl | 6.96* | Gel at 10 min | Gel after 30 min |
| 240 μl | 6.14* | Gel at 10 min | Gel at 10 min |
| 260 μl | 5.58 | | |
| | (gel) | | |

TABLE 11
enzyme dependent gelation with TCEP

| Amount of | | | |
|---|---|---|---|
| 32% HCl | | | |
| added | | | |
| (accumulated) | pH | +mTG | +buffer |
| — | 9.27 | — | — |
| 20 μl | 9.09* | Liquid after 3 hr | Liquid after 3 hr |
| 40 μl | 8.91 | — | — |
| 60 μl | 8.75 | — | — |
| 80 μl | 8.58* | Liquid after 3 hr | Liquid after 3 hr |
| 100 μl | 8.26 | — | — |
| 120 μl | 7.90 | — | — |
| 140 μl | 7.81* | Gel after 60 min | Viscous liquid after 60 |
| | | | min, gel after 2 hr |
| 160 μl | 7.23* | Gel after 18 min | Liquid after 18 min, |
| | | | viscous liquid after 30 min |
| 180 μl | 6.65* | gel in 3 min | Liquid after 3 min, gel |
| | | | after 15 min |
| 200 μl | 6.08* | White gel in 3 min | White gel in 3 min |
| 220 μl | 5.70 | — | — |
| 240 μl | 5.56 | — | — |
| | (gel) | | |

TABLE 12
enzyme dependent gelation with beta-mercaptoethanol

| Amount of | | | |
|---|---|---|---|
| 32% HCl | | | |
| added | | | |
| (accumulated) | pH | +mTG | +buffer |
| — | 10.69 | — | — |
| 20 μl | 10.52 | — | — |
| 40 μl | 10.34 | — | — |
| 60 μl | 10.09 | — | — |
| 80 μl | 9.92 | — | — |
| 100 μl | 9.74 | — | — |
| 120 μl | 9.48 | — | — |
| 140 μl | 9.20 | — | — |
| 160 μl | 8.76* | Semi-gel at 12 min, | Liquid after 2 hr |
| | | gel at 45 min, | |
| 180 μl | 8.02* | Gel at 12 min | Viscous liquid at 12 min, |
| | | | gel at 2 hr |
| 200 μl | 6.60 | | |
| | (gel) | | |

The above experiments show that at room temperature in the presence of reducing agent heat and urea denatured BSA can be kept in a liquid form above pH 5.5-6.5. The effective pH at which mTG is active is up to pH of about 9. Above room temperature, e.g. 37 C, mTG is more active but the solutions tend to undergo physical gelation. The pH range at which denatured BSA is in a liquid form, mTG is active and mTG dependent gelation is faster than physical gelation is preferably between 6 and 10 and more preferably between 7 and 9. Based on upon various experiments, urea is effective in a range of from 3M to 6M. As described in other experiments, the expected temperature range for maximum efficacy is 22 C to 37 C.

### Example 12

### Urea or Heat Alone are not Sufficient for mTG Dependent Gelation

Urea as a Denaturing Agent is not Enough for EDG (Enzyme Dependent Gelation):

5% BSA and various concentrations of urea (1.5, 3, 4.5, 6M) at 37° C. in the presence of mTG did not result in crosslinking. This result is unexpected based on the teachings of Totosaus A. et al (International Journal of Food Science and Technology, 2002. 37:589-601) about urea induced gelation. First, urea itself is not sufficient for physical gelation of BSA. Second, one would expect the denaturing of BSA induced by urea would suffice for EDG, but data shown herein demonstrates that both urea and disulphide reducing agent are required for EDG.

Urea is Necessary for EDG:

5% BSA in water that was heated at 70 C followed by TCEP (to dissolve the precipitate) did not gel with mTG. However, when the BSA contained 6M urea, a gel was formed.

### Example 13

### mTG Dependent Gelation Using Different Concentrations of Urea and/or BSA

The solutions below were prepared and heated at 70° C. for 10 minutes until white aggregates were formed. The aggregate was dissolved by adding 20 μl 4N NaOH and then the pH was adjusted to 8-9 with 6 μl HCl (32%). Although the aggregates were dissolved in all formulations, addition of HCl caused precipitation/aggregation of formulations A, B and C and they were discarded. Formulations D to H remained a liquid after HCl addition. To 300 μl of formulations D to H mTG or mTG buffer (20 mM Na citrate pH 6.0) were added, the tubes were incubated at 37° C. and gelation was determined by cessation of flow inside the tubes.

TABLE 13

| | BSA | Urea | DTT | | | | |
|---|---|---|---|---|---|---|---|
| formulation# | (%) | (M) | (mM) | pH | Physical state | +mTG | +mTG buffer |
| A | 5 | 1.25 | 25 | | Gel/aggregate | | |
| B | 5 | 2 | 25 | | Gel/aggregate | | |
| C | 5 | 3 | 25 | | Gel/aggregate | | |
| D | 5 | 6 | 25 | 8 | Clear liquid | Gel after over-night (5 ul) | Viscous liquid after |
| | | | | | | | over-night (5 ul) |
| E | 1 | 4.5 | 25 | 9 | Clear liquid | Liquid after over-night (5 | Liquid after over-night |
| | | | | | | or 15 ul) | (5 ul) |
| F | 3 | 4.5 | 25 | | Clear liquid | +5 ul: liquid after 5 hr, | +5 ul - liquid after 5 hr |
| | | | | | | +15 ul - gel within 1:45 hr | |
| G | 7.5 | 4.5 | 25 | 7.9 | 7.9 | +15 ul mTG: | +15 ul buffer: |
| | | | | | | 8 min: gel | 8 min: liquid |
| | | | | | | | 30 min: viscous liquid |
| | | | | | | | Over-night: gel |
| H | 10 | 4.5 | 25 | | Clear liquid | +15 ul mTG: | +15 ul buffer: |
| | | | | | | 8 min: viscous liquid | 1:20 hr: viscous liquid |
| | | | | | | 30 min: highly viscous | |
| | | | | | | liquid | |
| | | | | | | 1:20 hr: gel | |

The results above show that in order for denatured BSA to remain in a liquid form the concentration of urea should be greater than 3M and as described herein, preferably from 3M to 6M. The concentration of BSA should be from 1% to 10%.

### Example 14

### Effect of pH on BSA Denaturation

5% BSA containing 4.5M urea (pH 7) was titrated to the following pH values by adding HCl or NaOH. DTT was added to 25 mM. The results are shown in Table 14.

TABLE 14
Effect of pH on Denaturation

| pH | Appearance after 10 min at 70° C. |
|---|---|
| 2.9 | Solution remained clear |
| 4.0 | Solution remained clear |
| 5.4 | Solution turned into white gel w/aggregates |
| 6.0 | Solution turned into white gel w/aggregates |
| 7.0 | Solution remained clear |
| 9.7 | Solution remained clear |

600 ul 4N NaOH were added to the aggregate formed from BSA solution at pH 6.0 until the aggregate has dissolved, then the pH was adjusted to 8.0 with 2N HCl. To 300 μl of the resulting solution 15 μl mTG or mTG buffer (20 mM Na citrate pH 6.0) were added, the tubes were incubated at 37° C. and gelation was determined by cessation of flow inside the tubes. With mTG gel formed after 8 minutes, with buffer the solution was still a liquid after 50 minutes and a viscous liquid after 24 hours.

### Example 15

### Effect of Denaturation Temperature

1 ml solution of 5% BSA at pH 5 (Biological Industries, Israel), 4.5M urea and 25 mM DTT was heated for 10 minutes at either 50° C., 60° or 70° until white precipitation occurred. 20 μl of 4N NaOH was added to dissolve the precipitate followed by 5 μl HCl (32%).

300 μl of the dissolved precipitate were incubated at 37° C. with 15 μl of either mTG (33 IU/ml) or mTG buffer (20 mM Na citrate pH 6.0) and gelation was recorded as cessation of flow.

50° C.+mTG: viscous liquid after 50 min, gel after 24 hr

50° C.+buffer: liquid after 50 min, viscous liquid after 24 hr

60° C.+mTG: gel after 50 min

60° C.+buffer: liquid after 50 min, viscous liquid after 24 hr

70° C.+mTG: gel after 50 min

70° C.+buffer: liquid after 50 min, semi gel after 24 hr

### Example 16

### Effect of Urea, DTT and Heat Treatment on EDG

5% BSA solution (Sigma Aldrich), pH 7 was denatured with a combination of DTT, urea and heat (70° C. for 10 min) according to the following Table 15.

TABLE 15
Effect of Urea, DTT and heat treatment

| | | | Heat, | Physical |
|---|---|---|---|---|
| # | urea | DTT | 70° C. | state |
| 1 | no | 25 mM | yes | gel |
| 2 | no | 25 mM | no | liquid |
| 3 | 4.5M | 5 mM | yes | liquid |
| 4 | 4.5M | 10 mM | yes | liquid |
| 5 | 4.5M | 15 mM | yes | liquid |
| 6 | 4.5M | 25 mM | yes | liquid |
| 7 | 4.5M | no | yes | liquid |
| 8 | 4.5M | 25 mM | no | liquid |

Solution #1 became a gel during heating.

300 μl of solutions #1, 3 and 4 (after heating) were incubated at 37° C. with 15 μl of either mTG (33 IU/ml) or mTG buffer (20 mM Na citrate pH 6.0) and gelation was recorded as cessation of flow.

TABLE 16
Results of Gelation

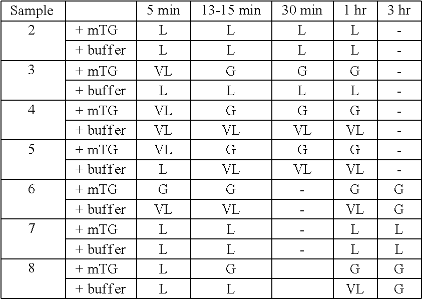

The results above show that without urea, EDG (Enzyme Dependent Gelation) does not occur when only DTT is present. When the mixture is heated at 70 deg the solution gels immediately, thus preventing addition of mTG to the denatured albumin (sample #1 in Table 16 above). Without heating the solution remained a liquid but addition of mTG did not result in EDG (sample #2). This is a surprising finding in light of the teachings of Kang et al which show that disulphide reducing agent is sufficient for mTG crosslinking of albumin

When the reaction contained urea but no DTT (sample #7) no EDG occurred as well, which corresponds to the previously described surprising results. When the reaction contained urea and DTT at different concentrations (5-25 mM), EDG was achieved (samples #3 to #6, respectively).

Pre-heating the sample at 70 deg (sample 6) facilitated EDG as it occurred at an earlier time point compared to when no heat was used for the same reaction (sample 8).

Heat treatment therefore may prevent EDG by causing an extensive denaturation that leads to physical gelation (sample 1) or facilitate EDG (sample 6).

For clarity, time-points where EDG was observed are filled gray in the above Table 16.

### Example 17

### Effect of Urea Concentration on EDG

5% BSA (pH 7) was mixed with urea and DTT according to Table 19 below and either subjected to heat treatment (70 deg for 10 min) or not. 300 μl of solutions that did not physically gel were incubated at 37° C. with 15 μl of either mTG (33 IU/ml) or mTG buffer (20 mM Na citrate pH 6.0) and gelation was recorded as cessation of flow. Table 17 shows the complete gelation results according to time.

TABLE 17
Effect of Urea Concentration on Gelation

| | | | | Physical |
|---|---|---|---|---|
| Sample | Urea | DTT | Heat, 70° C. | state |
| 1 | 1.5M | 5 mM | yes | liquid |
| 2 | 1.5M | 10 mM | yes | gel |
| 3a | 1.5M | 25 mM | yes | gel |
| 3b | 1.5M | 25 mM | no | liquid |
| 4 | 3M | 5 mM | yes | liquid |
| 5 | 3M | 10 mM | yes | Semi gel |
| 6a | 3M | 25 mM | yes | gel |
| 6b | 3M | 25 mM | no | liquid |
| 7 | 6M | 5 mM | yes | gel |
| 8 | 6M | 10 mM | no | liquid |
| 9 | 6M | 10 mM | yes | Semi gel |
| 10 | 6M | 25 mM | no | liquid |
| 11 | 6M | 25 mM | yes | liquid |

TABLE 18
Gelation Results

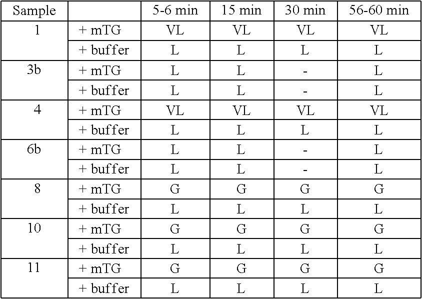

For clarity, time-points where EDG was observed are filled gray in the above Table 18.

The data presented above shows that EDG does not occur at pH<4.5 and that it does occur at urea concentrations as high as 6M.

The effect of heat treatment on denatured BSA physical state varies and depends on the concentrations of urea and/or DTT or the ratios between them or BSA.

### Example 18

### Effect of BSA Concentration on EDG

BSA (Sigma Aldrich, pH 7) at different concentrations (2.5%, 7.5% or 10%) containing 4.5M urea and different DTT concentrations (0 mM, 10 mM or 25 mM) was heated at 70° C. for 10 min or not heated. 300 μl of the resulting solution were incubated at 37° C. with 15 μl of either mTG (33 IU/ml) or mTG buffer (20 mM Na citrate pH 6.0) and gelation was recorded as cessation of flow. For clarity, samples where EDG was observed at any time-point are filled gray in the below Table 19.

TABLE 19
Effect of BSA concentration on gelation

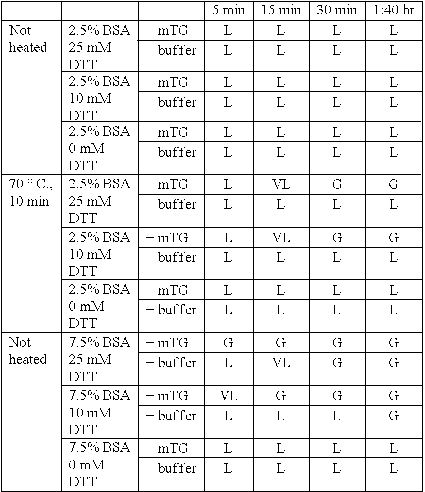

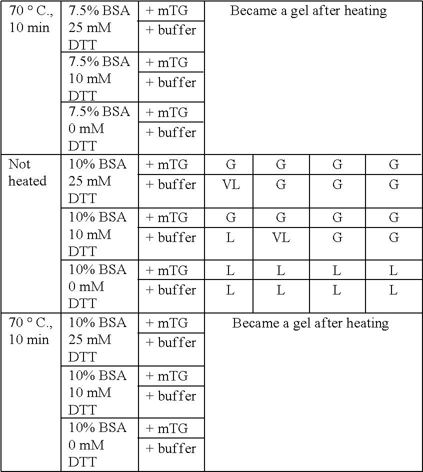

The results show that EDG occurs at all BSA concentrations tested (2.5%, 7.5% and 10%). EDG requires at least 5 mM DTT. Heat treatment was necessary for EDG at the lowest BSA concentration of 2.5%, but with higher BSA concentrations (7.5% and 10%) heat treatment cause immediate gelation, thereby preventing the addition of mTG. This demonstrates once again the importance of a balanced combination of all 3 denaturing treatments (disulphide reducing agent, chaotropic agent and heat) as well as BSA concentration and that such combinations cannot deduced apriori from known prior art. 1%-5% BSA, heat 50-90° C., 5-50 mM DTT, 3-6M urea or 5-10% BSA, no heat treatment, 5-50 mM DTT & 3-6M urea.

### Example 19

### Crosslinked Albumin Gel Properties

40 gr of a solution featuring 5% BSA (Sigma Aldrich), 6M urea, 25 mM DTT was mixed with 2 ml mTG solution (700 IU/ml). The resulting mixture was poured into three dog bone shaped molds, with 3.1 gram in each mold. Molds containing gels were incubated at 37° C. Within 60 minutes the mixture turned to a gel. The gels were covered with saline and incubated for 2 more hours and then were released from the molds.

For the testing of each gel, the tabs on either end of the dogbone shaped gel were clamped into a Model 3343 Single Column Materials Testing System (Instron™; Norwood, Mass.). The top tab was then pulled upwards at a rate of 0.5 mm/s, resulting in the creation of tensile force on the gel dogbone. Tension of sample was continued until failure was observed. Bluehill 2 Materials Testing Software (Instron™; Norwood, Mass.) was used to analyze results and calculate material properties including elastic modulus, peak stress, and strain to break. The results shown in Table 20 below demonstrate that crosslinked albumin gels are both strong and flexible, properties that make them suitable for medical applications.

TABLE 20
crosslinked albumin gel properties

| Property | Average + SD (n = 3) |
|---|---|
| Maximum load (N) | 0.31 ± 0.06 |
| Tensile stress at | 15.31 ± 2.79 |
| Maximum load | |
| (kPa) | |
| Tensile strain at | 19.35 ± 0.65 |
| maximum load (%) | |
| Young's modulus | 78.94 ± 12.56 |

### Brief Summary of Some Method Embodiments

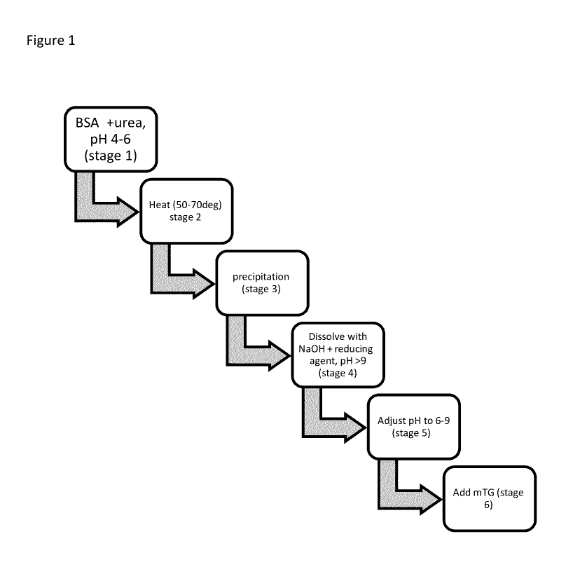

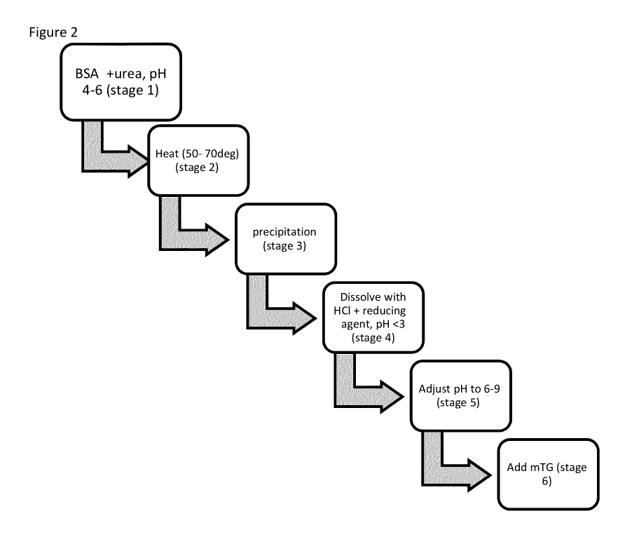

FIGS. 1 and 2 summarize some embodiments of at least some methods according to the present invention for cross-linking albumin. As shown in FIG. 1, in stage 1, BSA and urea are combined at pH 4-6. Heat is then applied in stage 2, preferably 50-70° C. The denatured protein then precipitates in stage 3. The precipitate is dissolved in stage 4 by raising the pH value (for example with NaOH) in the presence of a reducing agent, preferably at a pH value greater than 9. In stage 5 the pH is adjusted back to pH 7-9, after which transglutaminase, such as microbial transglutaminase, may be added in stage 6.

FIG. 2 shows a method in which stages 1-3 are identical to FIG. 1. However, in stage 4, the pH value is lowered (for example with HCl) and a reducing agent is applied, at a pH below 3. Stages 5 and 6 are again identical to FIG. 1.

Of course, the above methods of dissolving the precipitate may be avoided if the pH is maintained below 4 or above 6 at all times, and preferably at pH 6-9 (more preferably at pH 7-9) for the operation of transglutaminase to crosslink the albumin (BSA).

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A gel composition comprising denatured albumin, transglutaminase, a chaotropic agent and a disulfide bond reducing agent in a pH range of from 6 to 9.

2. The composition of claim 1, wherein said transglutaminase is calcium independent.

3. The composition of claim 2, wherein said transglutaminase is microbial transglutaminase.

4. The composition of claim 3, wherein a protein content of said transglutaminase is present in an amount from about 0.0001% to about 2% w/w of the composition.

5. The composition of claim 4, wherein said transglutaminase is present in an amount of from about 0.01% to about 1% w/w of the composition.

6. The composition of claim 5, wherein said transglutaminase is present in an amount of from about 0.1% to about 1% w/w of the composition.

7. The composition of claim 6, wherein said transglutaminase is present in an amount of from about 0.5% to about 1.5% w/w of the composition.

8. The composition of claim 1, wherein said disulfide bond reducing agent comprises one or more of a thiol containing reducing agent, dithiothreitol (DTT), cystein, glutathione, a phosphine-containing agent or a combination thereof.

9. The composition of claim 8 wherein said disulfide bond reducing agent comprises 2-mercaptoethanol.

10. The composition of claim 8, wherein said phosphine containing agent comprises tris(2-carboxyethyl) phosphine (TCEP).

11. The composition of claim 1, wherein said chaotropic agent is selected from the group consisting of urea, guanidinium chloride, and lithium perchlorate.

12. The composition of claim 11, wherein said urea is present in a range of from 3M to 6M.

13. The composition of claim 1, further comprising a salt.

14. The composition of claim 13 wherein said salt comprises sodium chloride.

15. The composition of claim 14, wherein said sodium chloride is present in an amount of from 0.25M to 1M.

16. The composition of claim 1, wherein said chaotropic agent comprises urea and wherein said disulfide bond reducing agent comprises at least one of TCEP, DTT or 2-mercaptoethanol.

17. The composition of claim 1, wherein said albumin is present in a concentration from 1% to 25%.

18. The composition of claim 17, wherein said albumin is present in a concentration from 1% to 10%.

19. The composition of claim 1, wherein said denatured albumin comprises heat denatured albumin.

20. The composition of claim 1, wherein a concentration of transglutaminase is in the range of from about 5 to about 200 enzyme units (U/g) of total composition.

21. The composition of claim 20, wherein said concentration of transglutaminase is in the range of from about 15 to about 55 enzyme units (U/g) of total composition.

22. The composition of claim 21, wherein said concentration of transglutaminase is in the range of from about 25 to about 45 enzyme units (U/g) of total composition.

23. A medical sealant, comprising the composition of claim 1.

24. A method of preparation of a gel composition, comprising: providing albumin with a pH value of from 4 to 6; heat denaturing said albumin to form denatured precipitated albumin; dissolving said denatured precipitated albumin with a disulfide bond reducing agent at a pH value of no more than 3 or of at least 9 to form reduced soluble albumin; adjusting a pH value of said reduced albumin to be between 6 and 9; and combining transglutaminase with said reduced albumin to form a combination.

25. The method of claim 24, further comprising applying a salt to said albumin to prevent precipitation.

26. A product produced according to the method of claim 25.

27. A method of treatment of a subject in need of treatment with a medical sealant or glue, comprising applying the product of claim 26 to a tissue of the subject as a medical sealant or glue.

28. The method of claim 27, wherein said applying comprises applying in a medical application selected from the group consisting of reinforcement of surgical repair lines; provision of fluid-stasis; prevention of lymphorrhea; prevention of cerebro-spinal fluid (CSF) leakage; prevention of anastomotic dehiscence; and sealing of an attachment between a tissue and a material.

29. The method of claim 27, wherein said medical sealant is provided in a form selected from the group consisting of a gel, a spray, a strip, a patch, and a bandage.

30. The method of claim 29, wherein said applying the product comprises applying the product in gel form as a medical glue.

31. Use of the composition of claim 1 for preparation of a tissue engineering scaffold.

32. A gel composition comprising heat denatured albumin, transglutaminase, a chaotropic agent and a disulfide bond reducing agent in a pH range of from 6 to 9.

33. A method of preparation of a gel composition, comprising: providing albumin with a pH value of from 6 to 9; denaturing said albumin with a chaotropic agent to form denatured albumin; treating said denatured albumin with a disulfide bond reducing agent at a pH value of 6 to 9 to form reduced albumin; and combining transglutaminase with said reduced albumin to form a combination.

34. The method of claim 33, further comprising adding a salt to said albumin to prevent precipitation before or during said denaturing.

35. The method of claim 33, wherein said denaturing said albumin comprises heat denaturing.

36. The method of claim 33, wherein said chaotropic agent comprises urea.
